(19) United States
(12) Patent Application Publication
(10) Pub. No.: US 2026/0216416 A1
(43) Pub. Date: Jul. 30, 2026

(54) APPARATUS FOR MITIGATION OF SURGICAL SMOKE

(71) Applicant: THE UNIVERSITY OF CHICAGO, Chicago, IL (US)

(72) Inventor: J. Michael MILLIS, Chicago, IL (US)

(73) Assignee: THE UNIVERSITY OF CHICAGO, Chicago, IL (US)

(21) Appl. No.: 19/144,998

(22) PCT Filed: Jan. 12, 2024
(86) PCT No.: PCT/US2024/011455

§ 371 Date: Jul. 1, 2025

Related U.S. Application Data
(60) Provisional application No. 63/480,242, filed on Jan. 17, 2023.
(60) Provisional application No. 63/595,550, filed on Nov. 2, 2023.

(51) Int. Cl. A61M 1/00; A61B 18/00; A61B 18/12

(52) U.S. Cl. CPC A61M 1/87; A61B 18/12; A61M 1/85; A61M 1/86; A61B 2018/00595; A61B 2218/008

(57) ABSTRACT

Apparatuses and methods for smoke mitigation during surgery. Some of such apparatuses comprising: a body extending around at least a majority of a perimeter of a medial region, the body defining an interior chamber, one or more openings facing the medial region and in fluid communication with the chamber, and a source connection in fluid communication with the chamber; where the body is configured to be coupled to a patient with the medial region overlying a surgical field, and the source connection is configured to be coupled to a vacuum source to draw air into the chamber through the one or more openings.

### CROSS-REFERENCED TO RELATED APPLICATIONS

This application claims the benefit of priority to each of (1) U.S. Provisional Patent Application No. 63/480,242, filed Jan. 17, 2023, and (2) U.S. Provisional Patent Application No. 63/595,550, filed Nov. 2, 2023; all of which foregoing applications are incorporated by reference in their respective entireties.

### FIELD OF INVENTION

The present invention relates generally to apparatuses and methods for surgical cauterization; and more specifically, but not by way of limitation, to an apparatus for aspirating smoke from a surgical field, for example smoke generated during electrocautery procedures.

### BACKGROUND

There has been increasing attention recently on potential issues related to smoke that generated by electrocautery devices during surgery. For example, the National Institute for Occupational Safety and Health (NIOSH) conducted a survey of more than 4,500 healthcare professionals to investigate exposure and mitigation of surgical smoke. Steege A L, et al., Secondhand smoke in the operating room? Precautionary practices lacking for surgical smoke, Am. J. Ind. Med., 59 (11): 1020-1031 (November 2016). The authors of at least one study have suggested that human papillomavirus (HPV) may be transmitted via surgical smoke from lasers. Hallmo P, et al., Laryngeal papillomatosis with human papillomavirus contracted by laser surgery. Eur. Arch. Otorhinolaryngol, 248:425-427 (1991). And several state legislatures have passed laws requiring evacuation of surgical smoke.

These developments have led to the creation of several systems for evacuating surgical smoke. Examples include the Neptune SafeAir, available from Stryker; and the MEGADYNE® Smoke Evacuator System, available from Ethicon. Use has also increased of wound protectors that protect tissue within a surgical incision, including limiting tissue from being exposed to surgical smoke. Examples of such surgical protectors are the Alexis line of surgical protectors available from Applied Medical.

### SUMMARY

While certain systems have been proposed or made available for mitigating or aspirating surgical smoke, at least some such systems incorporate a vacuum source into an electrocautery device and thereby require users to adapt to the new electrocautery device.

The present disclosure includes devices and methods for mitigation (i.e., aspiration or evacuation from a surgical field and/or adding positive pressure gas flow into the surgical field) of surgical smoke generated during surgery, such as via cauterization or laser incisions or excisions. The present mitigation devices include a body that at least partially encircles a surgical field and draws surgical smoke away from the surgical field and into the body of the device, from which the surgical smoke can be routed to a filter.

In some configurations of the present apparatuses for smoke mitigation during surgery, the apparatus comprises: a body extending around at least a majority of a perimeter of a medial region, the body defining an interior chamber, one or more openings facing the medial region and in fluid communication with the chamber, and a source connection in fluid communication with the chamber; where the body is configured to be coupled to a patient with the medial region overlying a surgical field, and the source connection is configured to be coupled to a vacuum source to draw air into the chamber through the one or more openings.

In some configurations of the present apparatuses, the body is partially or entirely flexible (e.g., resilient). In other configurations, the body is partially or entirely rigid.

In some of the foregoing configurations of the present apparatuses, the body defines a closed path fully surrounding the medial region.

In some of the foregoing configurations of the present apparatuses, each of the one or more openings has an elongated shape.

In some of the foregoing configurations of the present apparatuses, each of the one or more openings has a circular shape.

In some of the foregoing configurations of the present apparatuses, the chamber extends along the entirety of the closed path.

In some of the foregoing configurations of the present apparatuses, the body comprises a first body section and a second body section configured to be coupled to the first body section.

In some of the foregoing configurations of the present apparatuses, the apparatus further comprises: a first extension section coupled between the first and second body sections; and a second extension section coupled between the first and second body sections.

In some of the foregoing configurations of the present apparatuses, the second body section is configured to be slidably coupled to the first body section such that the size of the medial region can be changed by moving the second body section relative to the first body section.

In some of the foregoing configurations of the present apparatuses, the chamber is a first chamber, the one or more openings is one or more first openings, the source connection is a first source connection, and the body of the apparatus further defines an interior second chamber, one or more second openings facing the medial region and in fluid communication with the second chamber, and a second source connection in fluid communication with the second chamber. In some configurations, each of the one or more second openings has an elongated shape. In some configurations, the one or more first openings comprise a plurality of first openings that are spaced from one another along a first portion of the perimeter. In some configurations, the one or more second openings comprise a plurality of second openings that are spaced from one another along a second portion of the perimeter. In some configurations, the body second source connection is configured to be coupled to a positive-pressure source to deliver gas to the surgical field through the second opening(s). In other configurations, the body second source connection is configured to be coupled to a vacuum source to draw air into the chamber through the one or more second opening(s). In some such configurations, the first body section defines the first chamber and first openings and has the first source connection, and the second body section defines the second chamber and second openings and has the second source connection.

In some of the foregoing configurations of the present apparatuses, the minimum transverse dimension of the perimeter is greater than 100 millimeters (mm).

In some of the foregoing configurations of the present apparatuses, the source connection(s) comprise male tubing barbs.

In some of the foregoing configurations of the present apparatuses, the source connection(s) comprise quick connect fittings.

In some of the foregoing configurations of the present apparatuses, the body is coupled to a sterile surgical drape configured to extend outward relative to the medial region.

Some implementations of the present methods comprise: cauterizing tissue in a surgical field while one of the present apparatuses is coupled to a patient with the medial region overlying the surgical field and a vacuum source is coupled to the first source connection, such that smoke from the cauterization is drawn into the chamber through the opening(s).

Some implementations of the present methods comprise: cauterizing tissue in a surgical field while one of the present apparatuses with first and second source connections is coupled to a patient with the medial region overlying the surgical field, a vacuum source is coupled to the first source connection, and a positive-pressure source is coupled to the second source connection, such that gas exits the second opening(s) and pushes smoke from the cauterization toward the first opening(s) and the smoke is draw into the first chamber through the first opening(s). In some such implementations, the gas is inert. In other such implementations, the gas comprises air.

In some of the foregoing implementations of the present methods, the body comprises a first body second and a second body section, and the method further comprises: coupling, prior to cauterizing, the first body section to the second body section in the surgical field. In some such implementations, coupling the first body section to the second body section is completed via the first and second extension sections.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed configuration, the term “substantially” may be substituted with “within [a percentage] of” what is specified, where the percentage includes 0.1, 1, 5, and 10 percent.

Further, an apparatus or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any configuration of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one configuration may be applied to other configurations, even though not described or illustrated, unless expressly prohibited by this disclosure or the nature of the configurations. Additionally, while the present devices, apparatuses, and systems are described for certain applications (e.g., smoke mitigation in or around a surgical field), the present devices, apparatuses, and systems can also be used in other applications (e.g., non-surgical and/or non-medical settings, for example, to mitigate smoke or fumes during repair procedures such as welding, soldering, grinding, polishing, and/or the like).

Some details associated with the configurations described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configuration depicted in the figures.

FIG. 1 is an upper perspective view of a first example of the present apparatuses for mitigation of surgical smoke.

FIG. 2 is an exploded cross-sectional view of the apparatus of FIG. 1.

FIG. 3 is a top view of a base of the apparatus of FIG. 1.

FIG. 4 is a top view of a second example of the present apparatuses for mitigation of surgical smoke.

FIG. 5 is a side view of the apparatus of FIG. 4.

FIG. 6 is a top cross-sectional view of the apparatus of FIG. 4.

FIG. 7 is a top view of the apparatus of FIG. 4 in combination with a surgical drape.

FIG. 8 is a side view of a wound protector with the apparatus of FIG. 4 defining an upper ring of the wound protector.

FIG. 9 is a conceptual block diagram of the apparatus of FIG. 4 in combination with a source of vacuum and a source of gas.

FIG. 10 is a lower plan view of a third example of the present apparatus for mitigation of surgical smoke.

FIG. 11 is a perspective view of the apparatus of FIG. 10.

FIG. 12 is a top plan view of a variation of the apparatus of FIGS. 10 and 11 with body extension sections joining the two body sections of the apparatus in an elongated configuration.

FIG. 13 is a top plan view of a fourth example of the present apparatuses for mitigation of surgical smoke.

FIG. 14A is a top plan view of a fifth example of the present apparatuses for mitigation of surgical smoke.

FIG. 14B is a top plan view of the apparatus of FIG. 14 with the apparatus shown in an expanded configuration.

### DETAILED DESCRIPTION

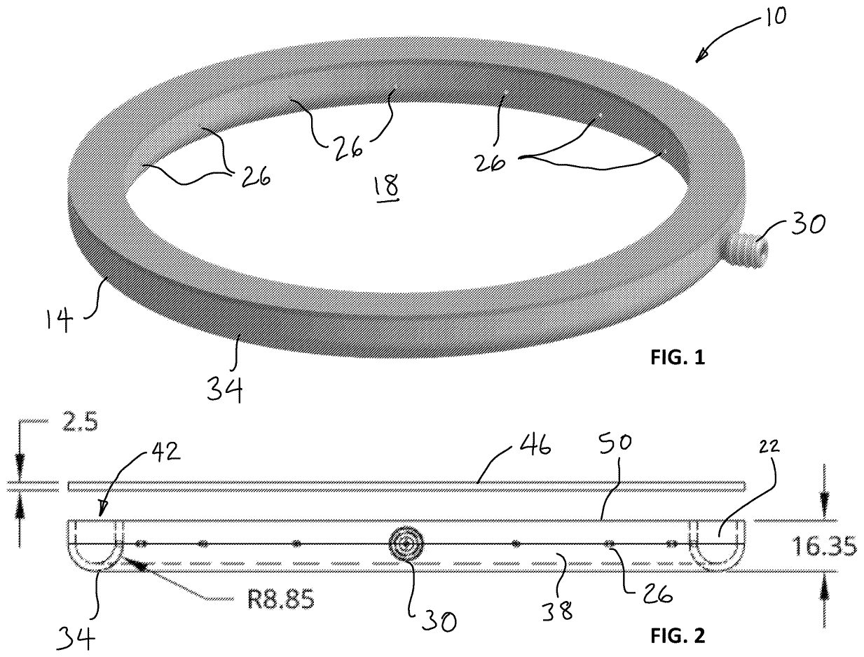

Referring now to the drawings, and more particularly to FIGS. 1-3, shown therein and designated by the reference numeral 10 is one example of the present apparatuses for smoke mitigation during surgery. In this example, the apparatus comprises a body 14 extending around at least a majority of a perimeter of a medial region 18; for example, by defining a closed path that fully surrounds the medial region 18 as shown (e.g., with chamber 22 extending along the entirety of that closed path, as shown). Body 14 defines an interior chamber 22, one or more (e.g., circular) openings 26 facing the medial region (18) and in fluid communication with chamber 22, and a source connection 30 in fluid communication with chamber 22. As shown, openings 26 can be spaced from one another along a first portion of the perimeter.

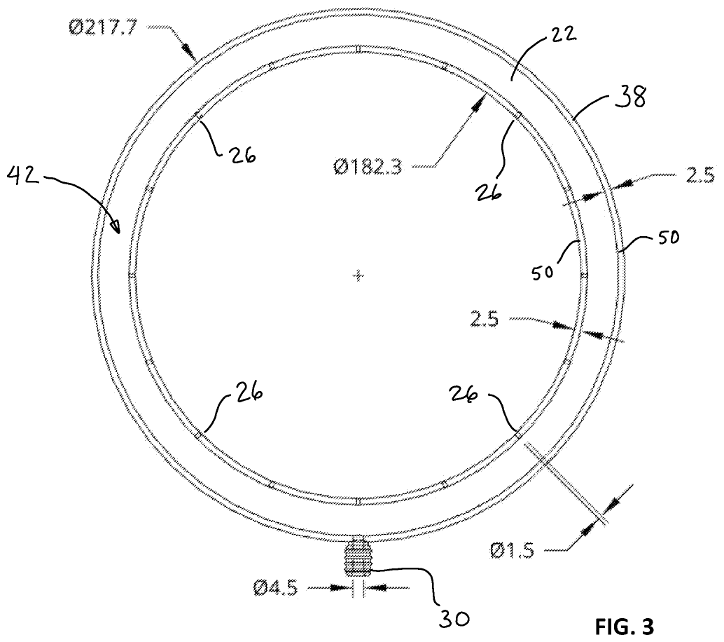

In this example, to facilitate manufacturing, body 14 comprises a U-shaped base 34 with a curved sidewall 38 defining a groove or channel with an open top 42 (in the depicted orientation), and a lid 46 configured to be coupled (e.g., sealed) to upper edges 50 of the sidewall (38) on either side of the channel to define the interior chamber (22) of the body. In the depicted example, source connection 30 comprises a nipple with male tubing barbs configured to be received in the end of a flexible tube, such as are commonly utilized to communicate vacuum in hospitals and surgical centers. Body 14 can comprise any suitable material, for example, polymer and/or metal alloys; and may, depending on material and dimensions, be resilient or substantially rigid. FIGS. 2 and 3 include dimensions in millimeters (mm) for one particular variation of the depicted apparatus 10, but are not limiting; apparatus 10 and others of the present apparatuses can be provided with any dimensions permitting the use of the present apparatuses for mitigation of surgical smoke in any of various surgical procedures for patients of any of various sizes (e.g., larger adults, smaller adults, children, and/or the like).

In use, body 14 is configured to be coupled to a patient with the medial region 18 overlying a surgical field (e.g., an incision in the patient's tissue), with source connection 30 coupled to a vacuum source to draw air into the chamber through openings 26. So arranged, apparatus 10 is configured to aspirate surgical smoke (e.g., generated by electrocauterization of tissue) from the surgical field by drawing the smoke through the openings (26) into the chamber (22), from which the smoke can be further drawn through the source connection 30 and through a passive filter to remove contaminants, and/or through an active filter to kill any pathogens, as described in more detail below.

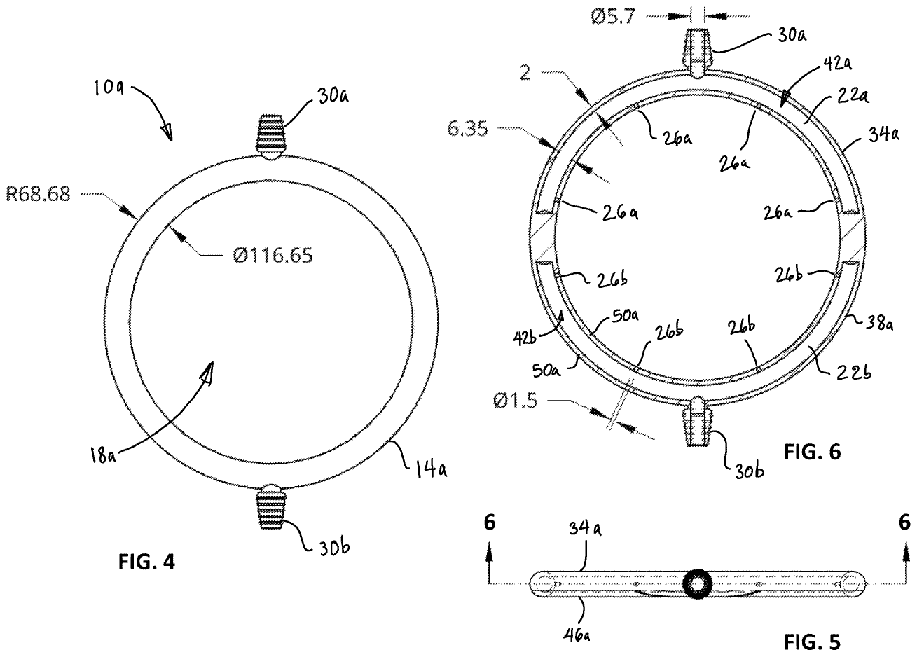

Referring now to FIGS. 4-6, shown there and designated by the reference numeral 10a is a second example of the present apparatuses for smoke mitigation during surgery. Apparatus 10a is similar to apparatus 10 in several respects. For example, apparatus 10a comprises a body 14a extending around at least a majority of a perimeter of a medial region 18a; for example, by defining a closed path that fully surrounds the medial region 18a as shown. Body 14a defines an interior chamber 22a, one or more (e.g., circular) openings 26a facing the medial region (18a) and in fluid communication with chamber 22a, and a source connection 30a in fluid communication with chamber 22a.

As with body 14 of apparatus 10, to facilitate manufacturing, body 14a of apparatus 10a comprises a U-shaped top 34a with a curved sidewall 38a defining a groove or channel with an open bottom 42a (in the depicted orientation), and a lid 46a configured to be coupled (e.g., sealed) to lower edges 50a of the sidewall (38a) on either side of the channel to define the interior chamber (22a) of the body. As with source connection 30 of apparatus 10, source connection 30a comprises a nipple with male tubing barbs configured to be received in the end of a flexible tube, such as are commonly utilized to communicate vacuum in hospitals and surgical centers. Body 14a can also comprise any suitable material, for example, polymer and/or metal alloys; and may, depending on material and dimensions, be resilient or substantially rigid. FIGS. 4 and 5 include dimensions in millimeters (mm) for one particular variation of the depicted apparatus 10a, but are not limiting; apparatus 10a and others of the present apparatuses can be provided with any dimensions permitting the use of the present apparatuses for mitigation of surgical smoke in any of various surgical procedures for patients of any of various sizes (e.g., larger adults, smaller adults, children, and/or the like).

However, apparatus 10a also differs from apparatus 10 in that body 14a of apparatus 10a also defines an interior second chamber 22b, one or more second openings 26b facing the medial region (18a) and in fluid communication with the second chamber (22b), and a second source connection 30b in fluid communication with the second chamber (22b). In this example, first openings 26a comprise a plurality of first openings that are spaced from one another along a first portion of the perimeter, and second openings 26b comprise a plurality of second openings 26b that are spaced from one another along a second portion of the perimeter. In the depicted example, second source connection 30b also comprises a nipple with male tubing barbs configured to be received in the end of a flexible tube, such as are commonly utilized to communicate vacuum in hospitals and surgical centers.

In use, body 14a is configured to be coupled to a patient with the medial region 18a overlying a surgical field (e.g., an incision in the patient's tissue), with first source connection 30a coupled to a vacuum source to draw air into the chamber through first openings 26a. So arranged, apparatus 10a is configured to aspirate surgical smoke (e.g., generated by electrocauterization of tissue) from the surgical field by drawing the smoke through first openings (26a) into the chamber (22a), from which the smoke can be further drawn through first source connection 30a and through a filter to remove contaminants, and/or through a sanitizing apparatus to kill any pathogens, as described in more detail below. Second source connection 30b can also be connected to a source of vacuum to aspirate surgical smoke (e.g., generated by electrocauterization of tissue) from the surgical field by drawing the smoke through second openings (26b) into the second chamber (22b), from which the smoke can be further drawn through second source connection 30b and through a filter to remove contaminants, and/or through a sanitizing apparatus to kill any pathogens. Alternatively, second source connection 30b can instead be coupled to a positive-pressure source (e.g., of air or an inert gas) to deliver gas to and/or into the surgical field through second openings 26b, for example, to urge surgical smoke toward the first openings (26a) and/or to dilute the smoke in the surgical field.

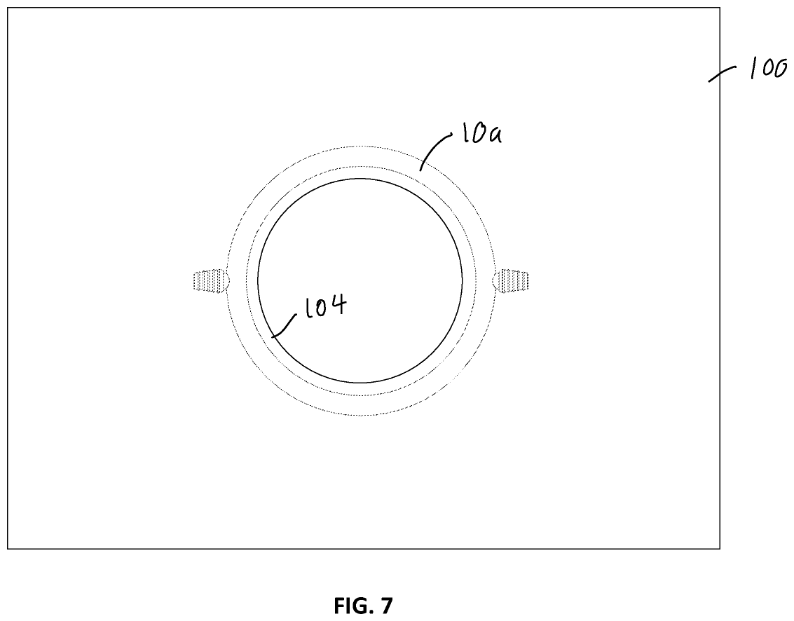

Referring now to FIG. 7, the present apparatuses (e.g., 10, 10a, 10b, 10c, 10d) can be coupled to a surgical drape 100 (e.g., with an adhesive bottom surface) to facilitate placement of the apparatus and maintaining sterility of a surgical field underlying the medial region (18a) of the apparatus. As shown, drape 100 can define an inner edge 104 encircling the medial region (18a), with edge 104 either pre-defined before placement of the drape and apparatus, or the drape can be cut to define edge 104 and the corresponding opening after the drape and apparatus have been positioned over the surgical field (e.g., to maintain sterility of the surgical field until a surgery begins and access is needed.

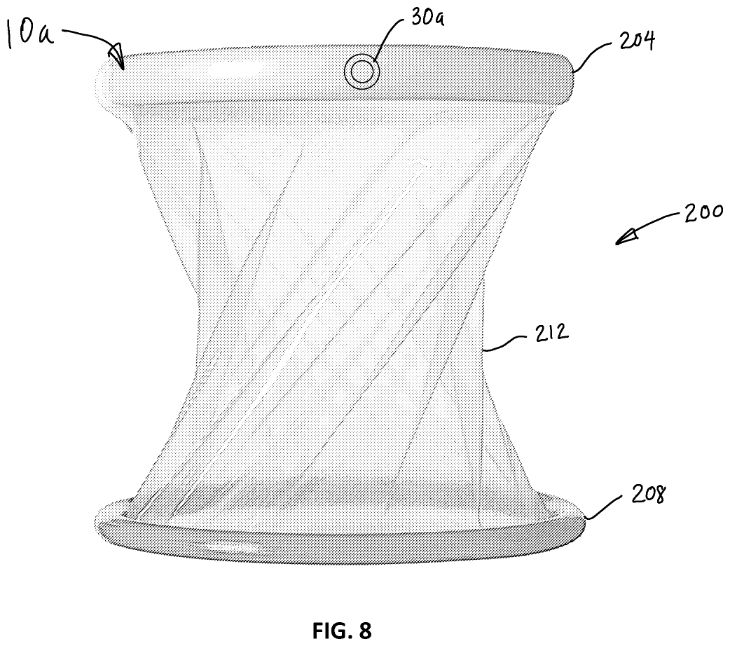

Referring now to FIG. 8, the present apparatuses (e.g., 10, 10a, 10b, 10c, 10d) can be coupled to a wound protector 200. More particularly, wound protector 200 is one example of a conventional wound protector having an upper ring 204, a lower ring 208, and a flexible barrier 212 extending between the upper and lower rings. In this example, apparatus 10a is included as the upper ring (204) such that barrier 212 assists with directing surgical smoke to the openings of the apparatus, or at least contains to some degree that surgical smoke to facilitate it being drawn through the openings of the apparatus. While the example of FIG. 8 shows apparatus 10a incorporated into the wound protector as the upper ring; in other configurations, apparatus 10a may be clipped or otherwise coupled to the upper ring of a conventional wound protector, may be simply disposed over a wound protector (e.g., with an annular skirt or drape extending radially outward from a perimeter of apparatus 10a).

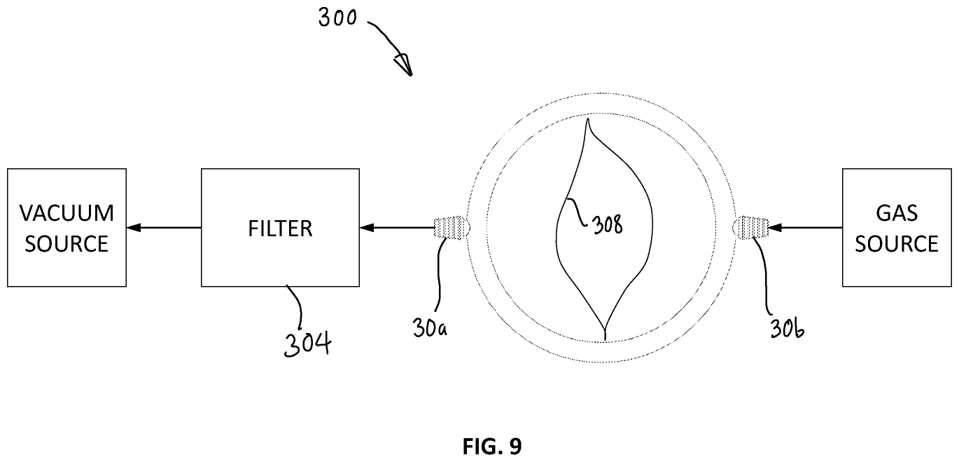

Referring now to FIG. 9, the present apparatuses (e.g., 10, 10a, 10b, 10c, 10d) can be incorporated into a system 300 also comprising a filtration device 304. Filtration device 304 can include one or more passive filters (e.g., a physical filter media) to remove contaminants from the smoke, one or more active elements (e.g., ultraviolet light) to kill pathogens in the smoke, or both one or more passive elements and one or more active elements. As shown, such filtration device 304 can be disposed between first source connection 30a and a vacuum source such as are commonly provided in hospitals and surgical centers. In other configurations, a local vacuum source may be incorporated into filtration device 304. Likewise, second source connection 30b can be coupled to a source of air or inert gas to cause such gas to flow out of second openings 26b to urge surgical smoke toward the first openings (26a) and/or to dilute the smoke in the surgical field, such as may arise from cauterization of tissue within a surgical incision 308.

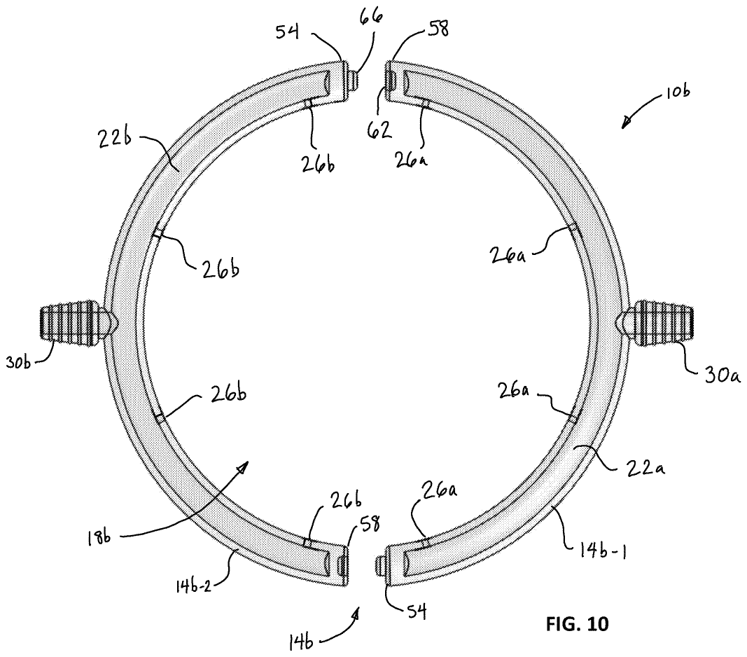

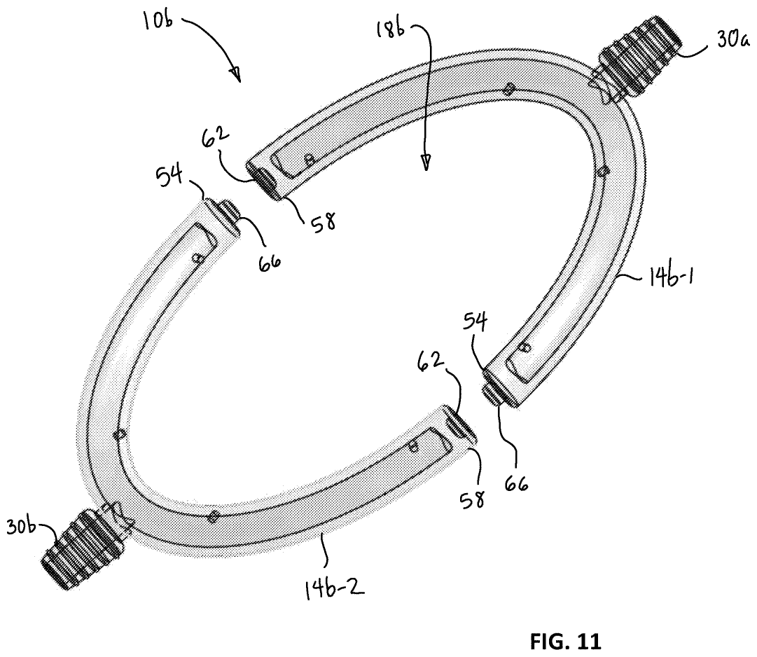

Referring now to FIGS. 10 and 11, shown there and designated by the reference numeral 10b is a third example of the present apparatuses for smoke mitigation during surgery. Apparatus 10b is similar to apparatus 10a in several respects. For example, apparatus 10b comprises a body 14b extending around at least a majority of a perimeter of a medial region 18b; for example, by—when assembled—defining a closed path that fully surrounds the medial region 18b as shown. Body 14b defines an interior first chamber 22a and an interior second chamber 22b, with one or more (e.g., circular) first openings 26a facing the medial region (18b) and in fluid communication with first chamber 22a, and a first source connection 30a in fluid communication with first chamber 22a. Body 14b also defines one or more second openings 26b facing the medical region (18b) and in fluid communication with second chamber 22b, and a second source connection 30b in fluid communication with second chamber 22b. As with apparatus 10a, in apparatus 10b, first openings 26a comprise a plurality of first openings that are spaced from one another along a first portion of the perimeter, and second openings 26b comprise a plurality of second openings 26b that are spaced from one another along a second portion of the perimeter.

As with apparatus 10a, in use, body 14b is configured to be coupled to a patient with the medial region 18b overlying a surgical field (e.g., an incision in the patient's tissue), with first source connection 30a coupled to a vacuum source to draw air into the chamber through first openings 26a. So arranged, apparatus 10a is configured to aspirate surgical smoke (e.g., generated by electrocauterization of tissue) from the surgical field by drawing the smoke through first openings (26a) into the chamber (22a), from which the smoke can be further drawn through first source connection 30a and through a filter to remove contaminants, and/or through a sanitizing apparatus to kill any pathogens, as described in more detail below. Second source connection 30b can also be connected to a source of vacuum to aspirate surgical smoke (e.g., generated by electrocauterization of tissue) from the surgical field by drawing the smoke through second openings (26b) into the second chamber (22b), from which the smoke can be further drawn through second source connection 30b and through a filter to remove contaminants, and/or through a sanitizing apparatus to kill any pathogens. Alternatively, second source connection 30b can instead be coupled to a positive-pressure source (e.g., of air or an inert gas) to deliver gas to and/or into the surgical field through second openings 26b, for example, to urge surgical smoke toward the first openings (26a) and/or to dilute the smoke in the surgical field.

Apparatus 10b differs from apparatus 10a in that body 14b includes a plurality of distinct sections that are configured to be coupled together to define medial region 18b. In the depicted example, body 14b includes a first section 14b-1 and a second section 14b-2. As shown, each section includes a first connector 54 on a first end of the section, and a second connector 58 on a second end of the section. In the depicted example, each second connector 58 defines a recess 62 that is sized and shaped to receive a corresponding protrusion 66 of a corresponding first connector 54 of an adjacent section with a press fit that resists separation of the connectors 54, 58. In other configurations, the connectors may utilize any type of fit (e.g., interference, barbed, grooved, or the like) that couples and thereby resists unintended separation of adjacent sections. While body 14b is shown with two sections; other configurations may have different numbers (e.g., 3, 4, or more) of sections, each defining an interior chamber and one or more openings facing the medial region 18b and in fluid communication with the interior chamber. Additionally, each section will include either a source connection (e.g., 18a, 18b) or will be configured to be coupled to an adjacent section to permit fluid communication between interior chambers of the coupled sections. For example, while connections 54 and 58 are solid such that chambers 22a and 22b remain distinct, other configurations may include passages through each of a respective pair of connections to enable fluid communication between interior chambers of two coupled sections (e.g., such that only one of the two coupled sections needs a source connector such as 30a or 30b).

In use, multi-section configurations (e.g., 10b) of the present apparatuses permit the apparatus to be placed adjacent to a surgical site as needed (e.g., while instruments are in and/or protruding from the site), rather than before surgical instruments are inserted into the site. For example, various surgical instruments (e.g., a clamp) may be needed in the site before a cautery is needed, and apparatus 10b may be assembled around the surgical site and any such instruments, rather than having to pass an apparatus over the ends of the instruments (which may be difficult or impossible, such as when an irrigation or suction line or the like is extending from the site). Specifically, body section 14b-1 and body section 14b-2 can be laterally moved together and the connectors 54, 58 pressed together to define medial region 18b around the surgical site. Likewise, when the need for smoke mitigation has passed (e.g., when cauterization is complete), body section 14b-1 and body section 14b-2 can be separated from each other and the apparatus removed from the surgical site, more readily and conveniently than with a single-piece apparatus.

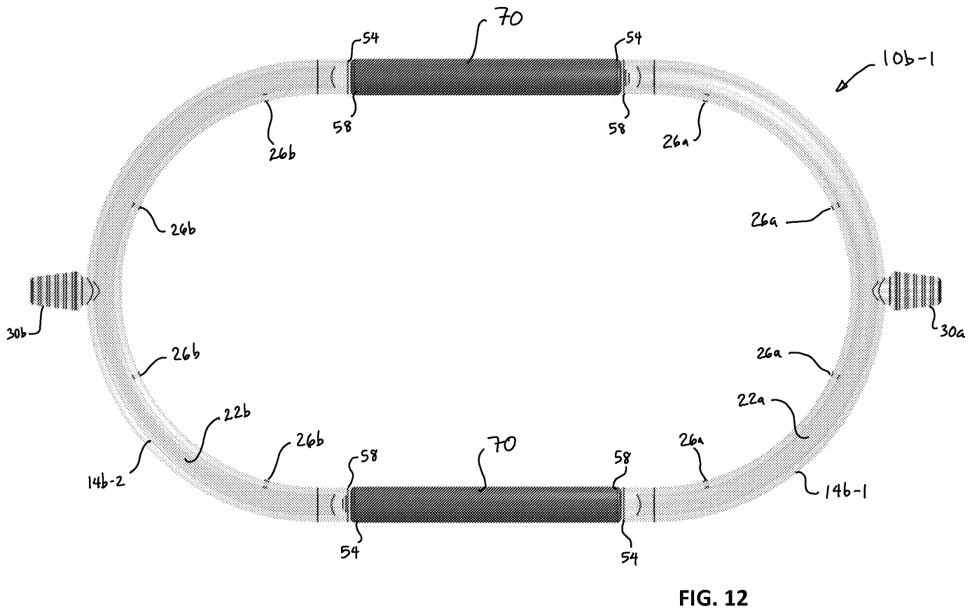

Referring now to FIG. 12, shown there and designated by the reference numeral 10b-1 is a variation of apparatus 10b in which extended connection segments 70 join the two body sections 14b-1, 14b-2 of the apparatus in an elongated configuration. In the depicted example, each extension section 70 is substantially straight and includes a first connector 54 on a first end of the section, and a second connector 58 on a second end of the section. As described above, each second connector 58 defines a recess 62 that is sized and shaped to receive a corresponding protrusion 66 of a corresponding first connector 54 of an adjacent section with a press fit that resists separation of the connectors 54, 58. Connectors 54, 58 of each extension section 70 are substantially similar to the respective connectors 54, 58 of each body section 14b-1, 14b-2 such that connector 54 of each extension section 70 will engage a connector 58 of either body section 14b-1, 14b-2, and such that connector 58 of each extension section will engage a connector 54 of either body section 14b-1, 14b-2. Such extension sections allow a provider to adapt the shape of body 14 for different sizes and/or shapes of surgical fields, for example, if a surgical field grows during a surgical operation. In the depicted configuration, extension sections 70 are solid such that gas or vacuum are not communicated through the extension sections. In other configurations, however, extension sections 70 may be hollow along all or some of their respective lengths and/or may include one or more openings through respective connector(s) 54 and/or 58, and/or may include one or more openings 26, 26a, 26b, to allow fluid communication through the respective extension section (e.g., to aspirate surgical smoke through the extension section to a corresponding body section 14b-1 or 14b-2).

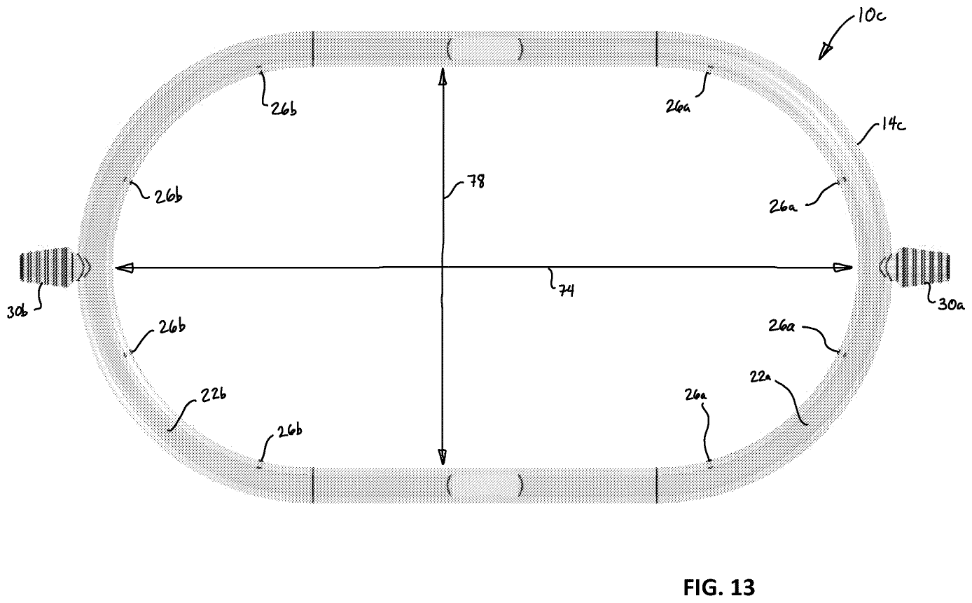

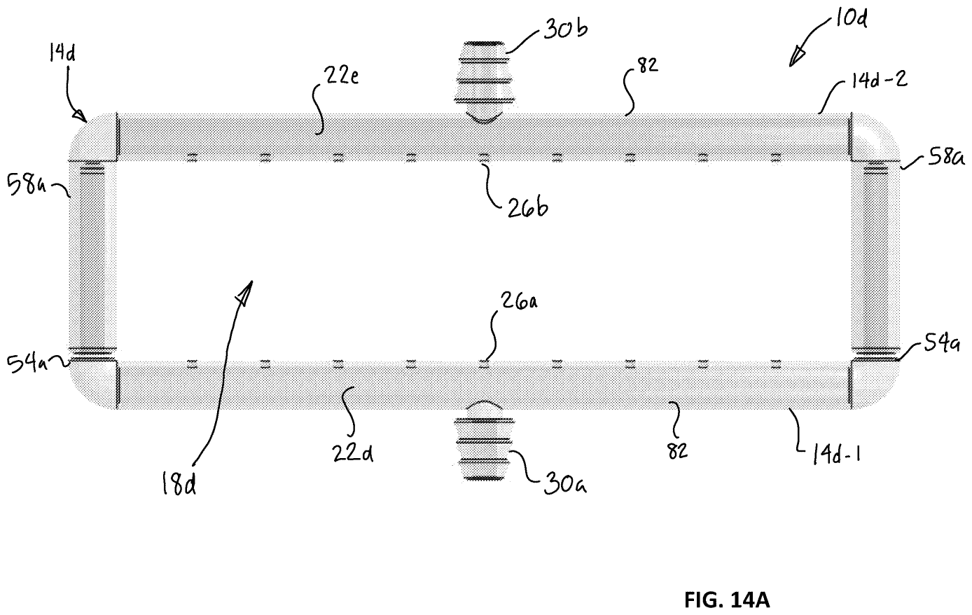

Referring now to FIG. 13, shown there and designated by the reference numeral 10c is a fourth example of the present apparatuses for mitigation of surgical smoke. Apparatus 10c is substantially similar to apparatus 10a, with the primary exception that body 14c has an elongated shape with an interior length 74 that is greater than (e.g., more than any one of, or between any two of: 100%, 110%, 120%, 130%, 140%, 150%, 160%, 170%, 180%, 190%, 200%, 250%, or more, of) interior width 78. The elongated shape of apparatus 10c permits body 14c to extend around surgical fields of different shapes, for example, a surgical field around an elongated incision.

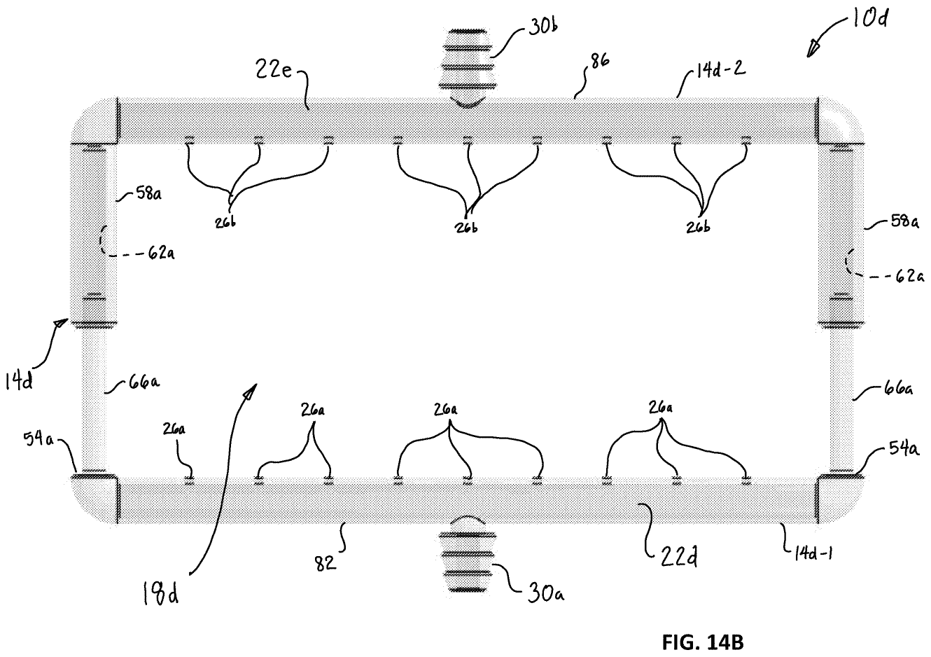

Referring now to FIGS. 14A and 14B, shown there and designated by the reference numeral 10d is a fifth example of the present apparatuses for mitigation of surgical smoke. Apparatus 10d is similar to apparatus 10b in several respects. For example, apparatus 10d comprises a body 14d extending around at least a majority of a perimeter of a medial region 18d; for example, by—when assembled—defining a closed path that fully surrounds the medial region 18d as shown. Body 14d defines an interior first chamber 22d and an interior second chamber 22e, with one or more (e.g., circular) first openings 26a facing the medial region (18d) and in fluid communication with first chamber 22d, and a first source connection 30a in fluid communication with first chamber 22d. Body 14d also defines one or more second openings 26b facing the medical region (18d) and in fluid communication with second chamber 22e, and a second source connection 30b in fluid communication with second chamber 22e. As with apparatus 10b, in apparatus 10d, first openings 26a comprise a plurality of first openings 26a that are spaced from one another along a first portion of the perimeter, and second openings 26b comprise a plurality of second openings 26b that are spaced from one another along a second portion of the perimeter.

As with apparatus 10d, in use, body 14d is configured to be coupled to a patient with the medial region 18d overlying a surgical field (e.g., an incision in the patient's tissue), with first source connection 30a coupled to a vacuum source to draw air into the chamber through first openings 26a. So arranged, apparatus 10d is configured to aspirate surgical smoke (e.g., generated by electrocauterization of tissue) from the surgical field by drawing the smoke through first openings (26a) into the chamber (22d), from which the smoke can be further drawn through first source connection 30a and through a filter to remove contaminants, and/or through a sanitizing apparatus to kill any pathogens, as described in more detail below. Second source connection 30b can also be connected to a source of vacuum to aspirate surgical smoke (e.g., generated by electrocauterization of tissue) from the surgical field by drawing the smoke through second openings (26b) into the second chamber (22e), from which the smoke can be further drawn through second source connection 30b and through a filter to remove contaminants, and/or through a sanitizing apparatus to kill any pathogens. Alternatively, second source connection 30b can instead be coupled to a positive-pressure source (e.g., of air or an inert gas) to deliver gas to and/or into the surgical field through second openings 26b, for example, to urge surgical smoke toward the first openings (26a) and/or to dilute the smoke in the surgical field.

As with body 14b of apparatus 10b, body 14d of apparatus 10d includes a plurality of distinct sections that are configured to be coupled together to define medial region 18d. In the depicted example, body 14d includes a first section 14d-1 and a second section 14d-2. However, apparatus 10d differs from apparatus 10b in that body section 14d-1 is slidably coupled to body section 14d-2 such that body 14d can be adjusted to change the size of medial region 18d between a compact configuration (FIG. 14A) and an expanded configuration (FIG. 14B). In the depicted example, first body section 14d-1 includes a straight primary segment 82 in which first chamber 22d is defined and through which first openings 26a are defined, and two connectors 54a each with an elongated piston projection 66a. In this example, second body section 14d-2 includes a straight primary segment 86 in which second chamber 22e is defined and through which second openings 26b are defined, and two connectors 58a each defining an elongated cylinder recess 62a. In other configurations, body sections 14d-1, 14d-2 may each include one connector 54a and one connector 58a. In either configuration, each elongated projection 66a is slidablye received in a corresponding elongated cylinder recess 62a such that body sections 14d-1, 14d-2 can slide apart and together to accommodate different or changing sizes of surgical fields (e.g., when an incision increases in size during a surgical procedure). In the depicted configuration, while connections 54a and 58a are solid such that chambers 22a and 22b remain distinct are are limited to respective primary segments 82 and 86; however, other configurations may include passages through each of a respective pair of connections to enable fluid communication between interior chambers of two coupled sections (e.g., such that only one of the two coupled sections needs a source connector such as 30a or 30b).

As with apparatus 10b, the multi-section configuration of apparatus 10d permits apparatus 10d to be placed adjacent to a surgical site as needed (e.g., while instruments are in and/or protruding from the site), rather than before surgical instruments are inserted into the site. For example, various surgical instruments (e.g., a clamp) may be needed in the site before a cautery is needed, and apparatus 10d may be assembled around the surgical site and any such instruments, rather than having to pass an apparatus over the ends of the instruments (which may be difficult or impossible, such as when an irrigation or suction line or the like is extending from the site). Specifically, body section 14d-1 and body section 14d-2 can be laterally moved together and the connectors 54a, 58a pressed together to define medial region 18d around the surgical site. If an incision increases in size during the surgery or additional space is needed to accommodate additional surgical tools, body sections 14d-1, 14d-2 can be moved apart toward the expanded configuration (FIG. 14B). Likewise, when the need for smoke mitigation has passed (e.g., when cauterization is complete), body section 14d-1 and body section 14d-2 can be separated from each other and the apparatus removed from the surgical site, more readily and conveniently than with a single-piece apparatus.

### Example & Experimental Data

Certain experiments were performed to qualitatively explore the impact on relative effectiveness of certain adjustments to operational variables when utilizing apparatus 10a of FIGS. 4-6. In particular, four experiments were performed with the variables described.
Test 1—Control-no suction or air flow other than ambient/room air flow
Test 2—Suction on one side of the apparatus (out of source connection 30a), at a level of 520 mmHg.
Test 3—Suction on both sides of the apparatus (out of each of source connection 30a and source connection 30b), at a level of 520 mmHg.
Test 4—Suction on one side of the apparatus (out of source connection 30a) at a level of 520 mmHg on Neptune; and positive air flow on the other side of the apparatus (into source connection 30b) at a rate of 12 liters per minute (l/min).

Suction was provided by a Neptune 3 waste management system (Stryker). The air flow for Test 4 was produced by an “E” cylinder of CO₂ with a standard ball variable regulator. The suction and air flow sources were connected to respective source connections of the apparatus via standard 3/16 inch tubing.

Smoke was generated by using an electrocautery device on a piece of meat in the middle of and slightly below the apparatus (10a). The electrocautery device was a Valleylab FT10 energy platform available (Medtronic) set on 40 coag. Only coagulation was utilized (no cut), and was on constantly for the time period to maximize smoke generation.

An air quality measurement instrument was disposed approximately 10 inches above the point of smoke generation, and was kept stable with a tripod. The air quality measurement instrument was a BR Smart Series real time air quality measurement device (BLATIN Science and Technology). Fine particulate matter (PM) peak measurements were recorded in micrograms/m³, and total volatile organic compounds (TVOC) measurements were recorded in mg/m³. PM2.5 measurements were of particles 2.5 microns or smaller, and PM10 measurements were of particles less than 10 microns in size.

The results are shown in the table below. As shown, each of Test 2, Test 3, and Test 4 increased the amount of time to trigger an alarm on the air quality measurement device, and for Test 3 avoided an alarm at all and maintained the lowest Max TVOC, PM2.5, and PM10 levels.

| | Length of | | | | | |
|---|---|---|---|---|---|---|
| | smoke | time of max reading | | | | Alarm goes off time |
| | generation | interval from start of | | | | point (sec) from start |
| | (secs) | smoke | Max TVOC | PM 2.5 | PM 10 | of smoke |
| Control | 45 | 45 | 2.7 | 910 | 943 | 5 |
| Test 2 | 70 | 60 | 0.814 | 605 | 925 | 21 |
| Test 3 | 90 | 48 | 0.76 | 32 | 55 | NA |
| Test 4 | 160 | 60 | 0.875 | 195 | 250 | 40 |

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted, modified, or combined as a unitary structure, connections may be substituted, or both. For example, openings 26, 26a, 26b may take other shapes (e.g., elongated shapes with each opening extending around a portion of the perimeter).

Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. An apparatus for smoke mitigation during surgery, the apparatus comprising:
a body extending around at least a majority of a perimeter of a medial region, the body defining an interior chamber, one or more openings facing the medial region and in fluid communication with the chamber, and a source connection in fluid communication with the chamber;
where the body is configured to be coupled to a patient with the medial region overlying a surgical field, and the source connection is configured to be coupled to a vacuum source to draw air into the chamber through the one or more openings.

2. The apparatus of claim 1, where the body is flexible.

3. The apparatus of claim 2, where the body is resilient.

4. (canceled)

5. The apparatus of claim 1, where the body defines a closed path fully surrounding the medial region.

6. The apparatus of claim 1, where each of the one or more openings has an elongated shape.

7. (canceled)

8. The apparatus of claim 1, where the chamber extends along the entirety of the closed path.

9. The apparatus of claim 1, where the body comprises a first body section and a second body section configured to be coupled to the first body section.

10. The apparatus of claim 9, further comprising:
a first extension section coupled between the first and second body sections; and
a second extension section coupled between the first and second body sections.

11. The apparatus of claim 9, where the second body section is configured to be slidably coupled to the first body section such that the size of the medial region can be changed by moving the second body section relative to the first body section.

12. The apparatus of claim 9, where the chamber is a first chamber, the one or more openings is one or more first openings, the source connection is a first source connection, and the body further defines an interior second chamber, one or more second openings facing the medial region and in fluid communication with the second chamber, and a second source connection in fluid communication with the second chamber.

13. The apparatus of claim 12, where each of the one or more second openings has an elongated shape.

14. The apparatus of claim 12, where the one or more first openings comprise a plurality of first openings that are spaced from one another along a first portion of the perimeter, and the one or more second openings comprise a plurality of second openings that are spaced from one another along a second portion of the perimeter.

15. (canceled)

16. The apparatus of claim 12, where the body second source connection is configured to be coupled to a positive-pressure source to deliver gas to the surgical field through the second opening(s).

17. The apparatus of claim 12, where the second source connection is configured to be coupled to a vacuum source to draw air into the chamber through the one or more second opening(s).

18. (canceled)

19. The apparatus of claim 1, where the minimum transverse dimension of the perimeter is greater than 100 millimeters (mm).

20. (canceled)

21. (canceled)

22. The apparatus of claim 1, where the body is coupled to a sterile surgical drape configured to extend outward relative to the medial region.

23. A method comprising:
cauterizing tissue in a surgical field while an apparatus of claim 1, is coupled to a patient with the medial region overlying the surgical field and a vacuum source is coupled to the source connection, such that smoke from the cauterization is drawn into the chamber through the opening(s).

24. A method comprising:
cauterizing tissue in a surgical field while an apparatus of claim 12, is coupled to a patient with the medial region overlying the surgical field, a vacuum source is coupled to the first source connection, and a positive-pressure source is coupled to the second source connection, such that gas exits the second opening(s) and pushes smoke from the cauterization toward the first opening(s) and the smoke is draw into the first chamber through the first opening(s).

25. The method of claim 24, where the gas is inert.

26. (canceled)

27. The method of claim 24, further comprising:
coupling, prior to cauterizing, the first body section to the second body section in the surgical field.

28. (canceled)
